(19) United States
(12) Patent Application Publication
(10) Pub. No.: US 2026/0150855 A1
(43) Pub. Date: Jun. 4, 2026

(54) YOGURT AND YOGURT PRODUCTION

(71) Applicant: Khalifa University of Science and Technology, Abu Dhabi (AE)

(72) Inventors: Fawzi Banat, Abu Dhabi (AE); Mustapha Mbye, Abu Dhabi (AE); Adiba Akram, Abu Dhabi (AE); Abdelmoneim Ali, Abu Dhabi (AE); Barathi Kannan Kalian, Abu Dhabi (AE)

(21) Appl. No.: 19/221,930

(22) Filed: May 29, 2025

Related U.S. Application Data
(63) Continuation-in-part of application No. 19/078,611, filed on Mar. 13, 2025.
(60) Provisional application No. 63/726,860, filed on Dec. 2, 2024.

(51) Int. Cl. A23C 9/13; A23B 11/12; A23C 9/123; A23C 9/133; C12N 1/20; C12R 1/01

(52) U.S. Cl. CPC A23C 9/1322; A23B 11/12; A23C 9/1236; A23C 9/1315; A23C 9/133; C12N 1/205; A23C 2240/15; A23V 2400/413; C12R 2001/01

(57) ABSTRACT

A method to produce yogurt includes pasteurizing a volume of milk, adding at least one additive to form a milk mixture, wherein the at least one additive comprises camel whey protein hydrolysate, and fermenting the milk mixture. P. acidilactici bacteria may be added to the milk mixture for fermentation. Yogurt produced by the method includes active P. acidilactici bacteria. The P. acidilactici bacteria may be P. acidilactici MNL2 bacteria.

### CROSS-REFERENCE TO RELATED APPLICATIONS

This application is a continuation-in-part of U.S. application Ser. No. 19/078,611 filed Mar. 13, 2025, which claims priority to U.S. provisional application 63/726,860 filed Dec. 2, 2024, the contents of each are incorporated by reference herein.

This application is related to copending, commonly owned U.S. application entitled “Cheese and Whey Products and Methods for Making Cheese and Whey Products” (attorney file No. 4105.131USC1).

### TECHNICAL FIELD

The subject matter disclosed herein relates to yogurt and methods to produce yogurt.

### BACKGROUND

Camel milk has been consumed for centuries in areas where camels are native, such as the Middle East, North Africa, and some parts of Asia. For decades, pastoralists consumed only fresh camel milk as a gift for hosts. As a result, its selling was frequently frowned upon, and it was not regarded as a commodity. Furthermore, it did not undergo any processing, excluding fermentation, to extend its shelf-life under desert conditions. Camel milk is a relatively new addition to the market, nationally or internationally. A more varied range of dairy products could be marketed due to the camel milk market's growth and the increasing understanding of its exquisite composition and transformation processes. With the availability of recent research, camel milk—a significant product recognized for its purported or real “medicinal” benefits—can now advance past its marginal status. Indeed, the camel milk market is forecasted to grow by US$1.88B during 2023-2028, accelerating at a compound annual growth rate of 5.17% during the prediction period.

Camel milk is well-recognized for its exceptional composition and nutritional aspects, making it an interesting substitute for bovine milk. It has attracted attention in the global market, and the demand for camel milk products has increased due to potential health aspects. As a result, camel dairies have been established in several countries, with certain products accessible, such as pasteurized milk, cheese, yogurt, and milk chocolates. The growing consumer interest and unique properties of camel milk make it a fascinating role market within the dairy industries. Camel milk is celebrated for its exceptional nutritional profile and superior digestibility compared to cow milk, positioning it as a premium ingredient in the dairy industry. For example, camel milk contains between 2.1 and 4.9% protein, comprising of casein (around 80% of the protein content) and whey protein (WP) (around 20% of the protein content) while bovine milk contains about 3.3% whey protein (WP). However, commercializing camel milk products, such as yogurt, faces challenges related to the milk's inherent compositional properties. Accordingly, camel milk is a convincing choice for individuals seeking healthy and functional foods. The lower cholesterol content in camel milk is also attractive for consumers. Thus there is a need for improved methods for the production of yogurt from camel milk.

It is known that traditional heat treatments cause chemical alterations of milk components, including denaturation of protein, loss of flavors and vitamins, nonenzymatic browning, and depression at freezing point, which might cause adverse variations in nutritional value, flavor, and color of milk. Heat processing is applied to guarantee a high safety level and shelf-life extension for dairy products. The heat treatment includes thermization (57-68° C. for 5-30 seconds), low-temperature long-time (63-66° C. for 30 min), high-temperature long-time (72° C. for 15 seconds), in-container sterilization at 110-115° C. for 10-20 min, and sterilization at ultra-high temperature (UHT) (130-140° C. for 3-5 seconds). Pasteurized and thermized milk are distributed in chilled conditions at 4-6° C., and high-temperature long-time pasteurized milk can be stored in a refrigerator for 10-14 days. UHT-treated milk may be stored at ambient temperature and can be kept for 6 months. Pasteurization inhibits psychotropic bacteria; however, heat-resistant extracellular enzymes (lipases and proteases) retain their activity throughout processing and storage, which might cause degradation of the treated product, including flavor defects and technological problems such as gelation and sedimentation.

Pasteurization is commonly employed with camel milk. However, the conditions of pasteurization applied by each holder are regularly decided without considering camel milk specificity, with the conditions being mostly based on the standards adjusted for bovine milk pasteurization. The reported conditions for camel milk pasteurization in the literature are fairly variable (60° C./30 min; 75° C./15 min; 63° C./30 min). Simultaneously, several private companies in the United Arab Emirates (UAE), Morocco, Algeria, Tunisia, Saudi Arabia, Mauritania, Kazakhstan, and Niger produce pasteurized camel milk. All of these companies apply different conditions for pasteurization. It is worth noting that regional/national/international standards for camel milk have not yet been established or have been adopted from bovine milk. In some countries, no standards are set by the authorities, or at least, it is proposed to use the same conditions applied for bovine milk pasteurization. UV irradiation has been previously utilized only on solid foods. The US Food and Drug Administration (FDA) and the US Department of Agriculture (USDA) have approved for use in liquid foods as an alternative to heat pasteurization. UV treatment has the benefits of low costs of installation, operation, maintenance, and energy usage. UV treatment also provides the benefits of preserving the textural, nutritional, and sensory attributes of the milk, not generating waste heat, producing no toxins or chemical residues, and it can be applied with other processing methodologies for synergistic or additive effects. Nevertheless, it has restrictions for use in opaque or cloudy liquids such as milk because of its low penetrating power. Also, prolonged exposure to UV might cause damage to humans (skin cancer, burns, eyes).

### SUMMARY

In one aspect, a method producing yogurt includes pasteurizing a volume of milk: adding at least one additive to form a milk mixture, wherein the at least one additive comprises camel whey protein hydrolysate, and fermenting the milk mixture.

In another aspect, a method of producing yogurt includes pasteurizing a volume of milk; adding at least one additive to form a milk mixture; and fermenting the milk mixture after adding P. acidilactici bacteria to the milk mixture, wherein the P. acidilactici bacteria are non-hemolytic and are characterized by one or more of: a survival rate in pH≤2.5 of approximately 86%-96%; a survival rate in pepsin of approximately 66%-76%; or a survival rate in bile salts and pancreatin of approximately 65%-75%.

In a further aspect, a fermented milk product includes active P. acidilactici bacteria that are non-hemolytic and characterized by one or more of: a survival rate in pH≤2.5 of approximately 86%-96%; a survival rate in pepsin of approximately 66%-76%; or a survival rate in bile salts and pancreatin of approximately 65%-75%. The fermented milk product may be yogurt.

### BRIEF DESCRIPTION OF THE DRAWINGS

FIG. 1 is a flowchart of a method to manufacture yogurt from milk, according to some embodiments.

FIG. 2 is a flowchart of a method to heat treat a volume of milk, according to some embodiments.

FIG. 3 is a schematic diagram of a system for antimicrobial treatment of camel milk using ultraviolet light according to some embodiments.

FIG. 4 is a flowchart of a method for antimicrobial treatment of camel milk using ultraviolet light according to some embodiments.

FIG. 5 is a schematic diagram of a system for antimicrobial treatment of camel milk using ultrasonic vibrations according to some embodiments.

FIG. 6 is a flowchart of a method for antimicrobial treatment of camel milk using ultrasonic vibrations according to some embodiments.

FIGS. 7A and 7B are graphs 702 and 704 illustrating data from a sensory evaluation (appearance, flavor, texture, overall acceptability) of camel milk yogurt (CM, control), camel milk yogurt fortified with whey powder (CM&WP), and bovine milk yogurt (BM) conducted on day 1 (graph 702), and day 15 (graph 704), using a 5-point hedonic scale. The scale ranges from 1 (dislike extremely) to 5 (like extremely), with intermediate scores of 2 (dislike slightly), 3 (acceptable), and 4 (like slightly).

FIG. 8 is a graph 800 illustrating results of the ability of different strains of lactic acid bacteria (LAB), isolated from a fermented herbal medicine product, to tolerate/survive acid, pepsin, and bile & pancreatin. Values are expressed as the mean±standard deviation (n=3).

FIGS. 9A and 9B illustrate a SDS gel image 902 and a graph 904 illustrating changes in the expression pattern of fat genes of worms exposed different diets. The image 902 and graph 904 illustrate that the worms' gene expression (three delta-9 desaturase homologs: fat-4, fat-5, and fat-6) differed when exposed different diets: OP50 (control); MNL2+Glucose; OP50+Glucose. Graph 904 illustrates the results of the gene expression for each diet. Values are expressed in mean±standard deviation (n=3). Different superscripts (a, b, c, d) represent significantly different values (p<0.05).

### DETAILED DESCRIPTION

The present disclosure describes a composition of yogurt and methods for the production of yogurt. Different types of milk that may be utilized with the methods disclosed herein include bovine (cow), sheep, goat, pig, mouse, water buffalo, camel, yak, horse, donkey, llama, or human milk. People who are sensitive or have allergies to bovine milk are particularly interested in yogurt products made from non-bovine milk. Because it is more challenging to produce yogurt from camel milk than other types of milk, the discussion of the methods disclosed herein focus on utilizing camel milk but other types of milk may be utilized to produce yogurt from the methods disclosed herein. For example, the inventors found that that they could not produce yogurt with properties acceptable to the consumer (appearance, texture, flavor, and storage) using the methods developed to make yogurt from bovine milk (e.g., temperature, ingredients). The differences in composition of non-bovine milk and bovine milk affect the production of yogurt and/or the properties of yogurt produced from non-bovine milk. For example, camel milk has larger micelle sizes; a low κ-casein-to-β-casein ratio; a diverse array of whey proteins; and higher proteolytic activity. The κ-casein-to-β-casein ratio is 0.05 for camel milk versus 0.33 for bovine milk. The differences in composition of non-bovine milk and bovine milk affect the production of yogurt and/or the properties of yogurt produced from non-bovine milk. The methods disclosed herein for yogurt production address challenges encountered in the production of high-quality fortified yogurt that has enhanced sensory, textural, and functional properties compared to existing formulations. For example, the methods described herein produce yogurt with properties similar to bovine milk yogurt (e.g., appearance, texture, flavor, and storage) to which consumers are accustomed and contain supplements such as prebiotics and probiotics. Prebiotics utilized in the disclosed methods herein include bottle gourd seed extract, artichoke extract, fenugreek extract, and/or gum Arabic extract. These plant-based supplements may also enhance coagulation of the milk during yogurt production. Yogurt with these plant-based supplements may also have antimicrobial properties. Another supplement that may be utilized in the disclosed methods is camel whey protein hydrolysate powder. Compared to bovine whey protein, camel whey protein was found to increase protein levels of the yogurt and/or improve water retention and firmness of the yogurt. The inventors also found that the quality of camel milk yogurt produced by adding bovine casein during the yogurt production was less than the quality of bovine milk yogurt. Without being bound by theory, this improved performance may be because bovine whey protein and/or bovine casein is not compatible with camel milk. A probiotic utilized in the disclosed method for the production of yogurt may be Pediococcus acidilactici bacteria. The P acidilactici bacteria may have lipid-lowering and cholesterol reducing properties, which may make the yogurt beneficial for obesity management. The P acidilactici bacteria also exhibits a high survival rate in simulated gastrointestinal conditions (low pH, pepsin, bile salts and pancreatin for an exposure duration of 2-3 hours). The P. acidilactici bacteria may also enhance shelf life of the yogurt because it may inhibit the growth of pathobiological bacteria. The bacteria may be P acidilactici MNL2.

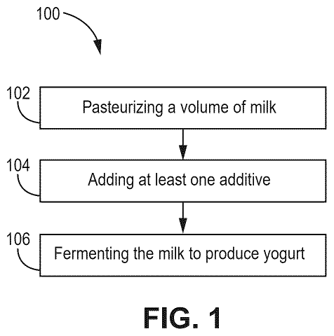

FIG. 1 is a flowchart of method 100 to produce yogurt from milk. In some embodiments, the yogurt is set yogurt. In other embodiments, the yogurt is stirred yogurt. The milk may be bovine, sheep, goat, pig, mouse, water buffalo, camel, yak, horse, donkey, llama, or human milk. Briefly, the method 100 includes pasteurizing a volume of milk at Step 102, adding at least one additive to form a milk mixture at Step 104, and fermenting the milk mixture at Step 106.

### Step 102 of Method 100—Pasteurizing a Volume of Milk

Pasteurizing a volume of milk at Step 102 may reduce the microbial load in the volume of milk. Thus, Step 102 may be described as an antimicrobial treatment. Types of pasteurization that may be utilized in Step 102 include ultraviolet (UV) light, ultrasonic pasteurization, cold plasma pasteurization, and/or thermal pasteurization. The method (e.g., temperatures, times) of pasteurizing milk for yogurt production may differ from pasteurizing milk for drinking. In some embodiments, thermal pasteurization is utilized at Step 102. In other embodiments, thermal pasteurization and either UV light or ultrasonic pasteurization is utilized. For example, pasteurizing the volume of milk may include applying UV light or ultrasonic pasteurization to the volume of milk and then heating the volume of milk to at least partially denature proteins in the milk. The method utilized to denature the proteins may be the same or similar to a thermal pasteurization method.

### Thermal Pasteurization

As noted above, in some embodiments, the volume of milk is thermally pasteurized at Step 102. A benefit of thermally pasteurizing milk in yogurt production is that the proteins in the milk may be at least partially denatured. This may improve fermentation (e.g., Step 106), the texture of the yogurt, and/or help prevent syneresis of the yogurt.

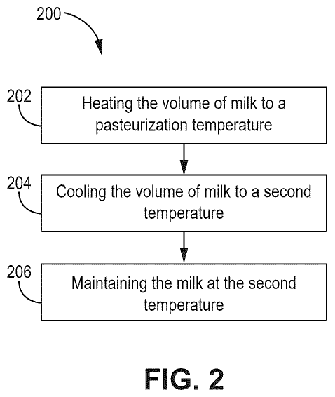

A flowchart of an exemplary thermal pasteurization method 200 for yogurt production is provided in FIG. 2. Briefly, the thermal pasteurization method 200 includes heating the volume of milk to a pasteurization temperature at Step 202, cooling the volume of milk to a second temperature at Step 204, and maintaining the milk at the second temperature at Step 206. Step 206 may temper the milk for Step 104 and/or Step 106 of method 100.

For Step 202, the volume of milk may be heated to the pasteurization temperature. The pasteurization temperature may be about 20%-32% less than the 85° C.-95° C. pasteurization temperatures that are typically used for yogurt production. For example, the pasteurization temperature may be 60° C.-70° C., 62° C.-70° C., 65° C.-70° C., 67° C.-70° C., 60° C.-67° C., 60° C.-65° C., 63° C.-67° C., less than 70° C., or at most 65° C. A temperature of the milk at the beginning of Step 202 may be about 4° C. In some embodiments, the rate at which the milk is heated from a beginning temperature to the pasteurization temperature is about 6° C. per minute to about 12° C. per minute, about 8° C. per minute to about 10° C. per minute, about 9° C. per minute, at least 6° C. per minute, or at most 12° C. per minute. In some embodiments, the milk is heated to the pasteurization temperature over a period of about 5 to 10 minutes, about 7 minutes, at least 5 minutes, or at most 10 minutes. At Step 202, the milk may be maintained at the pasteurization temperature for a pasteurization time period. The pasteurization time period may be 25-35 minutes, 27-33 minutes, 28-31 minutes, about 30 minutes, or at most 35 minutes. In one non-limiting example, the pasteurization temperature is 65° C. and the pasteurization time period is 30 minutes.

For Step 204, the milk may be cooled from the pasteurization temperature to a second temperature. The second temperature may be 33° C.-43° C., 35° C.-41° C., 37° C.-39° C., 33° C.-38° C., 35° C.-38° C., or at most 38° C. In one non-limiting example, the second temperature is 38° C. The milk may be cooled to the second temperature over a cooling time period. The cooling time period may be about 15-30 minutes, about 15-20 minutes, or at most about 30 minutes. In at least one embodiment, an ice water bath is utilized to cool the milk. However, other methods to cool the milk may be utilized.

For Step 206, the milk may be maintained at the cooling temperature for a third time period. The third time period may be 5-15 minutes, 7-13 minutes, 9-11 minutes, about 10 minutes, or at least 10 minutes. In one non-limiting example, the third time period is 10 minutes.

### UV Light Pasteurization

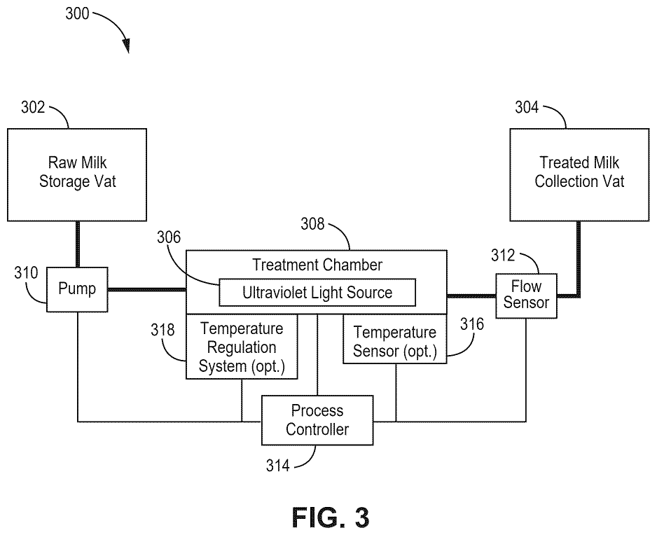

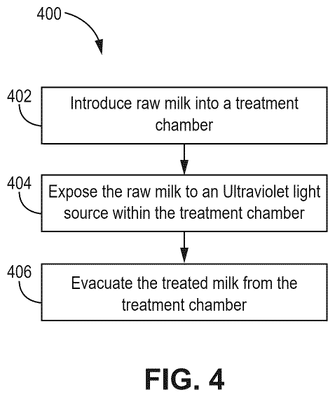

FIGS. 3 and 4 respectively illustrate a system 300 and a flowchart of a method 400 for UV light pasteurization, for example using the system 300, according to some embodiments. Turning to FIG. 3, the system 300 includes a first vat 302 for the storage of raw, untreated camel milk and a second vat 304 for the collection of treated milk after exposure to ultraviolet light generated by an ultraviolet light source 306 within a treatment chamber 308. The milk is moved from the first vat 302, through the treatment chamber 308, and into the second vat 304 by a pump 310 or other fluid movement device. In an alternative embodiment, the milk may be moved from the first vat 302, through the treatment chamber 308, and into the second vat 304 by a gravity induced flow. A flow rate of the pump 310 is preferably monitored by a flow sensor 312 and controlled by a process controller 314 to ensure adequate and consistent exposure of the camel milk to the ultraviolet light generated by the ultraviolet light source 306 within the treatment chamber 308. The flow rate of the pump 310 is preferably set so that the raw camel milk is exposed to the ultraviolet light source 306 within the treatment chamber 308 for at least 1.68 to 1.92 minutes.

The ultraviolet light source 306 provides ultraviolet light in the UV-C band having a wavelength in the range of 200-280 nanometers (nm). The ultraviolet light source 306 has a peak emission energy at about 254 nm. The ultraviolet light source 306 preferably has an intensity in the range of 4.1 to 5.5 milliwatts per square centimeter within the treatment chamber 308.

The system 300 may optionally include a temperature sensor 316 and a temperature regulation system 318, e.g. a refrigeration unit, interfacing with the process controller 314 to maintain a desired temperature of the various components of the system 300.

FIG. 4 is a flow chart of a method 400 for ultraviolet antimicrobial treatment of camel milk, for example using the system 300. This method 400 includes step 402 in which raw camel milk is withdrawn from a first vat 302 in which raw camel milk is stored and introducing the raw camel milk into a treatment chamber 308. The method 400 also includes step 404 in which the raw camel milk is exposed to an ultraviolet light source 306 within the treatment chamber 308. The method 400 further includes step 406 in which treated camel milk is evacuated from the treatment chamber 308 and collected in a second vat 304.

In the method 400, raw camel milk is continually introduced into the treatment chamber 308 and treated camel milk may be continually evacuated from the treatment chamber 308 due to the action of the pump 310. Introduction of camel milk into the treatment chamber 308 and evacuation of camel milk from the treatment chamber 308 is preferably performed at a constant flow rate in order to obtain consistent treatment of the raw camel milk. This constant flow rate may be obtained by monitoring the flow into or out of the treatment chamber 308 using a flow sensor 312 while controlling the flow through the pump 310 using a process controller 314 communicating with the flow sensor 312. The flow rate of the pump is preferably set so that the raw camel milk is exposed to the ultraviolet light source 306 within the treatment chamber 308 for at least 1.68 to 1.92 minutes. In another embodiment, the milk may be exposed for about 1.5 to about 3 minutes, about 1 to 4 minutes, about 1.7 to about 1.8 minutes, or about 1.6 to about 2 minutes.

The method 400 may optionally include regulating the temperature of the system 300 using a temperature sensor and a temperature regulation system, e.g., a refrigeration unit, interfacing with the process controller to maintain a desired temperature of the various components of the system 300. The method 400 is preferably conducted at a temperature of about 20° C. In another embodiment, the temperature may be in the range of about 15-25° C.

UV pasteurization of milk for drinking as discussed above may differ from UV pasteurization of milk for the production of yogurt. For example, the production of yogurt includes microbial, enzymatic, and physiochemical aspects not relevant to drinking milk. For example, although the raw-like flavor may be preserved and nutrients may be preserved by UV pasteurization, UV pasteurization may also have an oxidative effect on proteins and vitamins, especially B2, that may affect the texture, acidification, and/or nutrition of yogurt. As another example, overexposure to UV light could alter the casein micelles, for example modify micelle integrity, which could impair gel formation which may reduce yogurt firmness and syneresis control. As another example, residual LV exposure after inoculation could harm the bacteria added for fermentation at Step 106. For these reasons, UV pasteurization of milk for yogurt production may be performed at a slightly lower temperature, a reduced exposure time, and lower UV intensity to maintain the desired fermentability and curd-forming properties while reducing pathogenic microbes. An exemplary method for ultraviolet antimicrobial treatment that may be utilized for Step 102 includes exposing the volume of milk to ultraviolet light at a pasteurization temperature for a pasteurization time period. The pasteurization temperature may be 10° C.-15° C., about 16° C.-18° C., about 20° C., or at most 22° C. The pasteurization time period may be about 1 to about 1.8 minutes, about 1 to 4 minutes, about 1.6 to about 1.7 minutes, about 1.3 to about 2 minutes, at least 1.6 minutes, or at least 1.9 minutes. The wavelength of the UV light may be in the range of 190-260 nanometers (nm) (UVC light). The UV light source may have a peak emission energy at about 244 nm. The UV light source may have an intensity in the range of 3.8 to 5.1 milliwatts per square centimeter.

### Ultrasonic Pasteurization

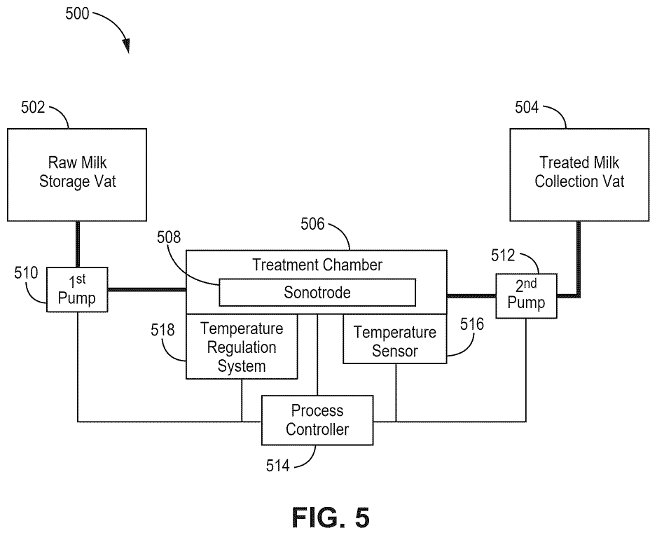

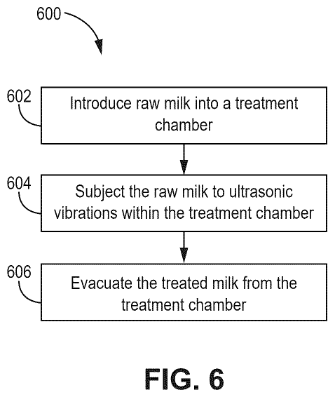

FIGS. 5 and 6 respectively illustrate a system 500 and a flowchart of a method 600 for ultrasonic antimicrobial treatment of camel milk, for example using the system 500 according to some embodiments. Turning to FIG. 5, the system 500 includes a first vat 502 for the storage of raw, untreated camel milk and a second vat 504 for the collection of treated milk after subjecting the raw camel milk to ultrasonic vibration within a treatment chamber 506 using a sonotrode 508. The milk is moved from the first vat 502 to the treatment chamber 506, and into the second vat 504 by a first pump 510 or other fluid movement device. Following treatment, the milk is moved from the treatment chamber 506 to the second vat 504 by a second pump 512. Operation of the first and second pumps 510, 512 is preferably controlled by a process controller 514 to ensure adequate and consistent exposure of the camel milk to the ultrasonic vibrations generated by the sonotrode 508 within the treatment chamber 506. The raw camel milk is exposed to ultrasonic vibrations within the treatment chamber 506 for about 10 minutes. In another embodiment, the time may be about 5 minutes to about 15 minutes. In an alternative embodiment, the milk may be moved from the first vat 502 to the treatment chamber 506 and from the treatment chamber 506 into the second vat 504 by a gravity induced flow where the first and second pumps 510, 512 are replaced by valves.

The ultrasonic vibrations preferably have a frequency of about 20 kHz (within typical tolerances) and a power of about 750 watts.

The system 500 includes a temperature sensor 516 and a temperature regulation system 518, e.g. a refrigeration unit, interfacing with the process controller to maintain a desired temperature of the treatment chamber 506 due to heat generated in the camel milk by the ultrasonic vibrations generating localized heat due to cavitation effects, when microscopic bubbles formed by the ultrasonic vibrations collapse and release energy, leading to a rise in temperature of the camel milk being treated in the treatment chamber 506.

FIG. 6 is a flowchart of a method 600 for ultrasonic antimicrobial treatment of camel milk, for example using the system 500. This method 600 includes step 602 in which raw camel milk is withdrawn from a first vat 502 in which raw camel milk is stored and the raw camel milk is introduced into a treatment chamber 506. The method 600 also includes step 604 in which the raw camel milk is subjected to ultrasonic vibrations within the treatment chamber 506 generated by a sonotrode 508. The method 600 further includes step 606 in which treated camel milk is evacuated from the treatment chamber 506 and collected in a second vat 504.

In the method 600, the camel milk is preferably treated in a batch process rather than a continuous process. The treatment chamber 506 is filled with raw camel milk from the first vat 502 and then treated by subjecting the milk to ultrasonic vibrations. The treated milk is then drained from the treatment chamber 506 to the second vat. Introduction of camel milk into the treatment chamber 506 and evacuation of camel milk from the treatment chamber 506 is preferably performed at a constant flow rate in order to obtain consistent treatment of the raw camel milk. The raw camel milk is subjected to the ultrasonic vibrations within the treatment chamber 506 for about 10 minutes. In another embodiment, the time may be between about 5 and about 15 minutes.

The sonotrode, configured to provide ultrasonic vibrations, operates at a frequency of 20 kHz and provides a power of about 750 watts. The ultrasonic vibrations may be applied in a pulsed mode with a 50% duty cycle for a period of at least 10 minutes. Each duty cycle may last 20 seconds and includes 10 seconds of applying the ultrasonic vibrations followed by 10 seconds of not the applying ultrasonic vibrations.

The method 600 may include regulating the temperature of the treatment chamber 506 using a temperature sensor 516 and a temperature regulation system 518, e.g., a refrigeration unit, interfacing with the process controller 514 to maintain a desired temperature within the treatment chamber 506. The method 600 is preferably conducted at a temperature of about 20° C.

As discussed above, the production of yogurt includes microbial, enzymatic, and physiochemical aspects that are not relevant to the production of drinking milk. Therefore, compared to ultrasonic pasteurization of milk for drinking, ultrasonic pasteurization of milk for yogurt production may be performed at a slightly lower temperature, a reduced pasteurization time period, and a reduced power level to maintain the desired fermentability and gel-forming properties while reducing pathogenic microbes. An exemplary method for ultrasonic antimicrobial treatment that may be utilized for Step 102 includes exposing the volume of milk to ultrasonic energy at a pasteurization temperature for a pasteurization time period. As discussed above, the ultrasonic energy may be characterized by a frequency and/or by power. The frequency may be about 20 kHz (within typical tolerances). The power may be about 730 watts, or at most 730 watts. The ultrasonic energy may be applied in a pulsed mode. The pulsed mode may be a 50% duty cycle. Each duty cycle may last 20 seconds and include 10 seconds of applying the ultrasonic vibrations followed by 10 seconds of not the applying ultrasonic vibrations. The pasteurization time period may be about 8 minutes or at least 8 minutes. The pasteurization temperature may be about 10° C.-18° C., about 18° C.-20° C., about 20° C., or at most 22° C.

### Step 104 of Method 100—Adding at Least One Additive to Form a Milk Mixture

In at least one embodiment, the at least one additive that may be utilized in step 104 for yogurt production includes camel whey protein hydrolysate powder. The camel whey protein hydrolysate may be a powder. The addition of camel whey protein hydrolysate was found to significantly improve the sensory attributes of camel milk yogurt, making it comparable to bovine milk yogurt in key qualities. For example, the camel whey protein hydrolysate powder may increase protein levels of the yogurt and/or improve water retention and firmness of the yogurt. In addition to camel milk, camel whey protein hydrolysate may be utilized for yogurt production from other types of milk. The at least one additive may further include one or more prebiotics and/or one or more coagulants. In some embodiments, the at least one additive that may be utilized in step 104 for yogurt production includes camel whey protein hydrolysate powder and bottle gourd seed extract. In other embodiments, the at least one additive for yogurt production includes camel whey protein hydrolysate (CWPH), bottle gourd seed extract, and artichoke extract. Bottle gourd seed extract and/or artichoke extract may function as prebiotics and/or as coagulants.

In at least one embodiment, 5% camel whey protein hydrolysate powder per 100 mL of pasteurized milk is added to the pasteurized milk. A 5% concentration of camel whey protein hydrosylate was determined to yield a yogurt structure that is comparable to bovine milk yogurt. However, other concentrations may be utilized. For example, the concentration of camel whey protein hydrosylate may be 3%-7%, 4%-6%, 3%-5%, or 5%-7% of the milk mixture. In one embodiment, 1% v/v/bottle gourd seed extract per 100 mL of pasteurized milk and 5% camel whey protein hydrolysate powder per 100 mL of pasteurized milk are added to the pasteurized milk. In another embodiment, 1% v/v artichoke extract per 100 mL of pasteurized milk, 1% v/v/bottle gourd extract per 100 mL of pasteurized milk; and 5% camel whey protein hydrolysate powder per 100 mL of pasteurized milk are added to the pasteurized milk. The concentration of bottle gourd seed extract and/or artichoke extract may be about 1% v/v per 100 mL, at least 1% v/v per 100 mL, or about 1%-2% v/v per 100 mL.

### Step 106 of Method 100—Fermenting the Milk Mixture

In at least one embodiment, Step 106 includes adding P. acidilactici bacteria to the milk mixture. Step 106 may include incubating the milk mixture at a fermentation temperature. In one non-limiting example, the fermentation temperature is 38° C. Step 106 may include fermenting until the fermentation mixture reaches a pH of approximately 4.3-4.6, approximately 4.5, or at most 4.6.

A strain of P. acidilactici suitable for yogurt production as disclosed herein may be characterized by one or more of the following: a survival rate of at least 10⁶ CFU/g, ability to survive in pH≤2.5, in pepsin, in bile salts and pancreatin, exhibit bile salt hydrolase (BSH) activity, exhibit cholesterol assimilation (thereby reducing the amount of cholesterol that may be absorbed by the host), exhibit antibiotic resistance to one or more antibiotics, or be non-hemolytic. The survival rate at the time of consumption may be at least 10⁶ CFU/g. The survival rate in pH≤2.5 may be approximately 86%-96%, at least 86%, at least 90%, at least 94%, at least 96%, or approximately 96%. The survival rate in pepsin may be approximately 66%-76%, at least 66%, at least 70%, at least 74%, at least 76%, or approximately 76%. The survival rate in bile salts and pancreatin may be approximately 65%-75%, at least 65%, at least 69%, at least 73%, or approximately 75%. The bile salt hydrolase (BSH) activity may be characterized by a precipitation zone of approximately 15 mm-17 mm, at least 15 mm, at least 17 mm, or approximately 17 mm. The exposure duration to evaluate the survival rate for low pH, pepsin, and/or bile salts and pancreatin may be 2-3 hours. The cholesterol assimilation may be approximately 80%-89%, at least 80%, at least 85%, at least 89%, or approximately 89%. The antibiotic resistance may include resistance to one or more of: ampicillin, streptomycin, penicillin, tetracycline, kanamycin, erythromycin, gentamicin, clindamycin, and chloramphenicol.

In at least one embodiment, the P. acidilactici strain is P. acidilactici MNL2. P. acidilactici MNL2 was found to be non-hemolytic and to exhibit the following characteristics: a survival rate of at least 10⁶ CFU/g, a survival rate in pH≤2.5 of approximately 96%, a survival rate in pepsin of approximately 76%, a survival rate in bile salts and pancreatin of approximately 75%, bile salt hydrolase (BSH) activity measured by a precipitation zone of approximately 17 mm, cholesterol assimilation of approximately 89%, and antibiotic resistance to ampicillin, streptomycin, penicillin, tetracycline, kanamycin, erythromycin, gentamicin, clindamycin, and chloramphenicol. Samples of P. acidilactici MNL2 were deposited on [date of the deposit with name and address of depository, accession number for the deposit].

### Experimental Assessments—Ultraviolet and Ultrasonic Pasteurization

Experiments were conducted to evaluate ultraviolet and ultrasonic treatment of camel milk, e.g., effect on microbial load, nutritional value and the longevity of the milk. The experimental results indicated that ultraviolet treatment was effective in reducing the microbial load of camel milk in one cycle (3-log reduction, 99.9%). Other treatments, including ultrasonic treatment, also achieved the standard microbial inhibition at 70% amplitude for 20 min and a power of 50% for 30 s, respectively. As shown in Table 1, heat treatment achieves a robust microbial reduction of 99.999% (typically 5-log) for most pathogens, slightly surpassing UV treatment, which provides a 99.9% reduction (typically 5-log).

TABLE 1
Comparative impact of Pasteurization vs. UV Treatment
on Microbial Pathogens in camel Milk

| | Heat-treated | UV Treatment |
|---|---|---|
| Microbial Pathogen | (72° C. for 15 sec) | (light) |
| Escherichia | 99.999% (5-log | 99.99% (5-log |
| coli | reduction) | reduction) |
| Salmonella | 99.999% (5-log | 99.9% (5-log |
| spp. | reduction) | reduction) |
| Listeria | 99.999% (5-log | 99.9% (5-log |
| monocytogenes | reduction) | reduction) |
| Staphylococcus | 99.999% (5-log | 99.9% (5-log |
| aureus | reduction) | reduction) |
| Total Bacterial | 99.9% reduction | 99.0-99.9% reduction |
| Count | | |

The chemical composition of camel milk was not significantly affected by UV treatment compared to thermal pasteurization, ultrasonication, and microwave. The activity of phosphatase and lactate dehydrogenase enzymes was also determined before and after processing. Additionally, the mineral profile in camel milk was evaluated before and after ultraviolet and ultrasonic treatment. The nutrients and various bioactive contents in camel milk were insignificantly affected by ultraviolet and ultrasonic as compared to thermal pasteurization. As shown in Table 2, heat treatment processing significantly diminishes bioactive components in camel milk, with reductions of 20% in lactoferrin, 17% in lysozyme, and 25% in immunoglobulin G. In contrast, UV treatment resulted in minimal reductions of 3%, 2.3%, and 2%, respectively. These findings underscore the potential of UV treatment as an effective processing method to preserve the nutritional and functional qualities of camel milk while maintaining microbial safety.

TABLE 2
Concentrations of Lactoferrin, Lysozyme, and Immunoglobulin G in
Camel Milk: Raw Milk, heat treated and UV-treated camel milk.

| | Raw | Heat-treated | UV-Treated |
|---|---|---|---|
| | Camel Milk | Camel Milk | Camel Milk |
| Component | (mg/L) | (mg/L) | (mg/L) |
| Lactoferrin | 180 | 140 | 175 |
| Lysozyme | 60 | 48 | 59 |
| Immunoglobulin G | 12 | 5 | 10 |

### Experimental Assessments—Yogurt

Experiments were conducted to compare yogurt made from camel's milk (CM), yogurt made from camel's milk supplemented with 5% Camel whey protein hydrolysate (CWPH) and 2% v/v bottle gourd seed extract (CW&WP) and yogurt made from bovine (cow) milk, with the yogurt made from bovine milk serving as a reference.

For example, titratable acidity, pH values, and water holding capacity (WHC) were compared. The observed acidity trends suggest that higher total solids content enhances buffering capacity and promotes acid production during storage as shown in (Table 3). The initial pH values ranged from 4.3 to 4.5, which is ideal for yogurt. By day 15, bovine milk (BM) yogurt maintained a stable pH (4.3), whereas camel milk (CM) yogurt experienced a slight reduction, reflecting increased acid production over time. Water Holding Capacity (WHC) measures the yogurt's ability to retain whey and is a quality indicator to minimize undesirable whey separation (syneresis). As can be seen in Table 3, the addition of whey powder to camel milk (CM) yogurt significantly enhanced Water Holding Capacity (WHC) in camel milk (CM) yogurt, reducing syneresis and improving its structural integrity. These results suggest that making yogurt from camel milk using method 100 produces yogurt with superior water retention and enhanced consumer appeal.

TABLE 3
Titratable acidity, pH values, and water holding
capacity (WHC) of experimented yogurt samples.

| | Titratable acidity | | | | | |
|---|---|---|---|---|---|---|
| | (% of lactic acid) | | pH | | WHC (%) | |
| Yogurt | Day 1 | Day 15 | Day 1 | Day 15 | Day 1 | Day 15 |
| CM | 1.0 ± 0.1 ^{aB} | 1.8 ± 0.4 ^{aA} | 4.2 ± 0.2 ^{aA} | 4.1 ± 0.3 ^{aA} | 31.5 ± 3.2 ^{cA} | 28.3 ± 1.0 ^{cB} |
| CM&WP | 1.0 ± 0.2 ^{aB} | 2.2 ± 0.3 ^{aA} | 4.4 ± 0.0 ^{aA} | 4.3 ± 0.1 ^{aB} | 71.5 ± 2.6 ^{aA} | 68.5 ± 1.4 ^{aB} |
| BM | 0.8 ± 0.2 ^{bB} | 1.2 ± 0.3 ^{bA} | 4.5 ± 0.1 ^{aA} | 4.3 ± 0.4 ^{aA} | 62.1 ± 4.2 ^{bB} | 58.1 ± 0.3 ^{bA} |

a-c means that different lowercase superscripts in the same column are significantly different for each parameter while A,B values with different uppercase superscripts in the same row significantly differ for each parameter.

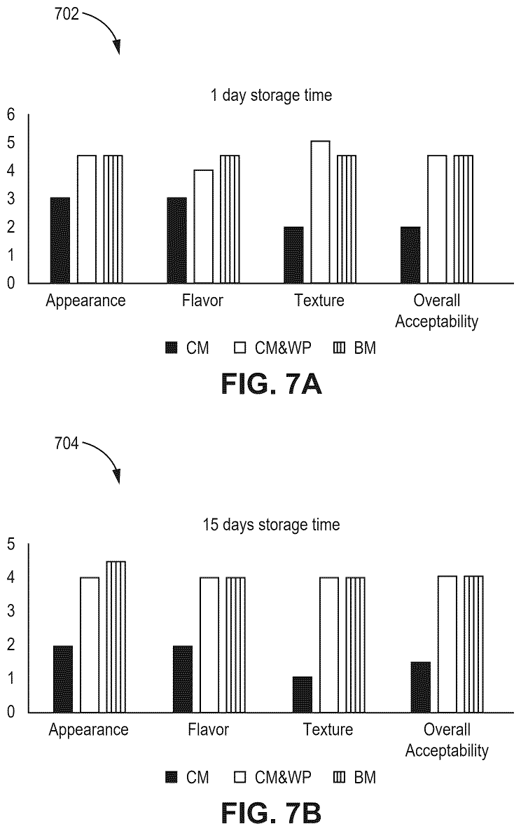

Experiments were conducted to evaluate textural characteristics of a camel milk control yogurt (CM), CM yogurt fortified with whey powder (CM&WP), and bovine milk (BM) yogurt over 15 days of storage (appearance, flavor, texture, overall acceptability). The results are graphically illustrated in FIGS. 7A and 7B (for each characteristic CM are the bars to the left; CM&WP are the middle bars; and BM are the bars to the right) and provided in Table 4 where a-c means that different lowercase superscripts in the same column are significantly different for each parameter while A,B values with different uppercase superscripts in the same row significantly differ for each parameter. Values are means±SD (n=6). As shown by graph 702 and graph 704 respectively provided in FIGS. 7A and 7B, whey powder fortification significantly improves the sensory attributes of camel milk control yogurt (CM), making it comparable to bovine milk (BM) yogurt in key qualities.

TABLE 4
textural characteristics of CM yogurt (control),
CM yogurt fortified with whey powder (CM&WP),
and BM yogurt over 15 days of storage

| | Yogurt | Storage period | |
|---|---|---|---|
| Parameter | treatment | Day 1 | Day 15 |
| Hardness | CM | 10.1 ± 0.8 ^{cA} | 7.4 ± 0.3 ^{cB} |
| (g) | CM&WP | 235.0 ± 3.1 ^{aA} | 161.6 ± 4.0 ^{aB} |
| | BM | 171.0 ± 5.0 ^{bA} | 111.5 ± 3.0 ^{bB} |
| Adhesiveness | CM | 0.11 ± 0.1 ^{cA} | 0.09 ± 0.1 ^{cA} |
| (mJ) | CM&WP | 1.3 ± 0.4 ^{bA} | 0.40 ± 0.1 ^{aB} |
| | BM | 1.2 ± 0.2 ^{aA} | 0.40 ± 0.0 ^{bB} |
| Resilience | CM | 0.74 ± 0.1 ^{aA} | 0.63 ± 0.0 ^{aA} |
| | CM&WP | 0.6 ± 0.0 ^{bB} | 0.3 ± 0.0 ^{bA} |
| | BM | 0.02 ± 0.0 ^{c A} | 0.04 ± 0.0 ^{cB} |

P. acidilactici MNL2—Isolation and Characterization

Moola Nivarana Lehyamis a traditionally fermented herbal medicine product, obtained from India, that is primarily used for obesity reduction. Moola Nivarana Lehyam comprises a blend of dried spices, including Medhya Rasayan (nootropic herb), Terminalia chebula (3.10%), Emblica officinalis (3.10%), Terminalia bellirica (3.10%), Withania somnifera (6.85%), Zingiber officinale (3.6%), Phytophthora megasperma (3.6%), Cyperus rotundus (7%), Tamarindus indica (7%), Aloe barbadensis miller (7%), Cissus quadrangularis (7%), Amorphophallus paeoniifolius (3%), Arenga pinnata (5%), and butter (10%).

Moola Nivarana Lehyam undergoes natural fermentation at 30° C. for 72 hours. Lactic acid bacterial (LAB) species were isolated and ten LAB strains were examined for bile salt hydrolase (BSH) activity and cholesterol assimilation (Table 5). All ten strains exhibited BSH activity, and their cholesterol assimilation capacity ranged from 10.2% to 89.5%. Among them, strain MNL2 demonstrated the highest cholesterol assimilation (89.3%) and the largest BSH activity zone (17 mm of precipitation). The isolated MNL2 strain was identified through 16S rRNA gene sequencing and exhibited a 99% similarity to Pediococcus acidilactici (GenBank accession: PP346234).

TABLE 5
Assimilation of cholesterol, Hemolytic activity
and BSH of isolated LAB strains

| | | | | | Cholesterol |
|---|---|---|---|---|---|
| Isolate | Color | | Hemolytic | BSH | assimilation |
| Name | morphology | Shape | activity | activity | (%) |
| MNL 1 | White/ | cocci | NA | ++ | 25.3 |
| | Smooth | | | | |
| MNL 2 | White | cocci | NA | +++ | 89.3 |
| MNL 3 | White/ | rod | NA | − | 10.2 |
| | Round | | | | |
| MNL 4 | White/ | rod | NA | − | 11.7 |
| | Smooth/ | | | | |
| | Round | | | | |
| MNL 5 | White/ | cocci | NA | +++ | 89.5 |
| | Smooth | | | | |
| MNL 6 | White | cocci | NA | + | 32.1 |
| MNL 7 | White | rod | NA | − | 25.5 |
| MNL 8 | White | cocci | NA | + | 23.4 |
| MNL 9 | White | cocci | NA | ++ | 41.5 |
| MNL 10 | White/ | rod | NA | − | 24.5 |
| | Round | | | | |

All isolated LAB strains were tested against nine antibiotics (Table 6 and Table 7), including ampicillin, streptomycin, penicillin, tetracycline, kanamycin, erythromycin, gentamicin, clindamycin, and chloramphenicol. Most isolates were susceptible to these antibiotics. However, P. acidilactici MNL2 displayed resistance to all nine antibiotics. The evaluation followed European Food Safety Authority (EFSA) guidelines, suggesting that P. acidilactici MNL2 could potentially contribute to human health benefits when administered alongside antibiotics.

TABLE 6
Antibacterial activity of Lactic Acid Bacteria (LAB)
cell free supernatant against standard pathogens.
(Zone of inhibition measured by millimeter—mm).

| | Protein synthesis inhibiting | | | |
|---|---|---|---|---|
| | targeting Antibiotic | | | |
| | Tetracycline | Kanamycin | Clindamycin | Erythromycin |
| Isolate | (30 μg · | (30 μg · | (30 μg · | (30 μg · |
| Name | L − 1) | L − 1) | L − 1) | L − 1) |
| MNL 1 | 24 | NZ | 22 | NZ |
| MNL 2 | 19 | 10 | 28 | 15 |
| MNL 3 | 12 | NZ | 25 | 14 |
| MNL 4 | 12 | 10 | 10 | 10 |
| MNL 5 | NZ | NZ | 14 | NZ |
| MNL 6 | 22 | 20 | 16 | 16 |
| MNL 7 | 18 | 12 | 24 | 14 |
| MNL 8 | 26 | 18 | 14 | NZ |
| MNL 9 | NZ | 18 | 12 | NZ |
| MNL 10 | 18 | NZ | 24 | 12 |

TABLE 7
Antibacterial activity of Lactic Acid Bacteria (LAB)
cell free supernatant against standard pathogens.
(Zone of inhibition measured by millimeter—mm

| | | | | Nucleic Acid |
|---|---|---|---|---|
| | Protein synthesis inhibiting | | | targeting |
| | targeting Antibiotic | | | Antibiotics |
| | Ampicillin | Novobiocin | Penicillin | Erythromycin |
| Isolate | (30 μg · | (30 μg · | (30 μg · | (30 μg · |
| Name | L − 1) | L − 1) | L − 1) | L − 1) |
| MNL 1 | 30 | 18 | 13 | NZ |
| MNL 2 | 20 | 18 | 30 | 15 |
| MNL 3 | 21 | 19 | 23 | 14 |
| MNL 4 | 18 | 18 | 20 | 10 |
| MNL 5 | 26 | 18 | 26 | NZ |
| MNL 6 | 28 | 12 | 12 | 16 |
| MNL 7 | 19 | 186 | 24 | 14 |
| MNL 8 | 13 | NZ | 11 | NZ |
| MNL 9 | 14 | 14 | 12 | NZ |
| MNL 10 | 24 | 20 | 13 | 12 |

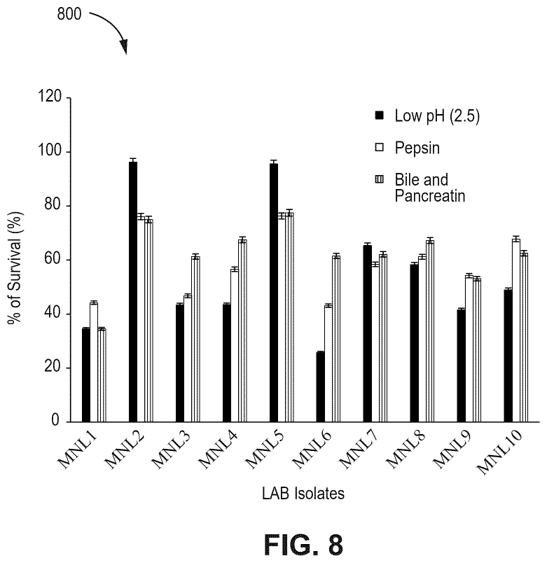

Experiments were conducted to evaluate the survival of P. acidilactici MNL2 under simulated gastrointestinal conditions that included exposure to low pH, pepsin, and/or bile salts and pancreatin for an exposure duration of 2-3.5 hours. The graph 800 provided in FIG. 8 illustrates the results. The MNL2 strain demonstrated high tolerance, with survival rates exceeding 96.3% in low pH, 76.1% in pepsin, and 75% in bile salts and pancreatin. These findings indicate the strain's significant resistance in simulated intestinal environments, supporting its probiotic potential.

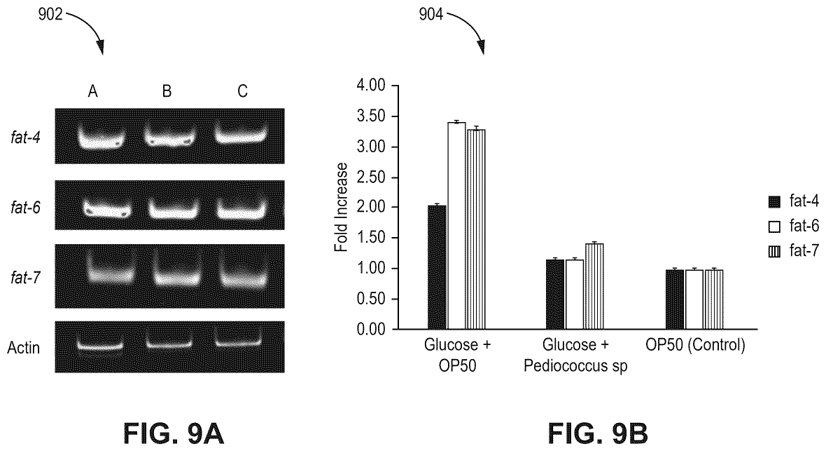

Experiments were conducted to evaluate body size alterations in Caenorhabditis elegans worms. C. elegans worms were monitored over three days under different dietary conditions. As illustrated by image 902 provided in FIG. 9A, the expression of three delta-9 desaturase homologs (fat-4, fat-5, and fat-6), which encode fatty acid desaturase enzymes responsible for introducing double bonds in saturated fatty acids, was downregulated in the MNL2+Glucose-treated group. This suggests that P. acidilactici MNL2 influences lipid metabolism through a mechanism that suppresses fatty acid desaturase gene expression, leading to reduced fat accumulation. As illustrated by graph 904 provided in FIG. 9B, worms fed a glucose+E. coli OP50 diet exhibited an increase in body size, while those on a glucose+MNL2 diet showed a reduction in size compared to the control (E. coli OP50 diet). These findings suggest that P. acidilactici MNL2 may play a role in modulating metabolic processes, potentially extending lifespan in the C. elegans N2 model. To investigate the impact of P. acidilactici MNL2 on lipid metabolism, RT-PCR analysis was conducted on the C. elegans N2 model.

While the invention has been described with reference to an exemplary embodiment(s), it will be understood by those skilled in the art that various changes may be made, and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment(s) disclosed, but that the invention will include all embodiments falling within the scope of the appended claims.

### Discussion of Possible Embodiments

The following are non-exclusive descriptions of possible embodiments of the present invention.

In some aspects, the techniques described herein relate to a method for ultraviolet antimicrobial treatment of camel milk, including introducing raw camel milk into a treatment chamber, exposing the raw camel milk to an ultraviolet light source within the treatment chamber, and evacuating treated camel milk from the treatment chamber.

The method of the preceding paragraph can optionally include, additionally and/or alternatively any, one or more of the following features/steps, configurations and/or additional components.

In some aspects of the method, the raw camel milk is continually introduced into the treatment chamber and the treated camel milk is continually evacuated from the treatment chamber.

In some aspects of the method, introduction of camel milk into the treatment chamber and evacuation of camel milk from the treatment chamber is performed at a constant flow rate.

In some aspects of the method, the ultraviolet light source provides ultraviolet light in the UV-C band having a wavelength in a range of 200-280 nanometers (nm).

In some aspects of the method, the ultraviolet light source has a peak energy emission at about 254 nm.

In some aspects of the method, the ultraviolet light source has an intensity in a range of 4.1 to 5.5 milliwatts per square centimeter.

In some aspects of the method, the flow rate allows the raw camel milk to be exposed to the ultraviolet light source for at least 1.68 to 1.92 minutes.

In some aspects of the method, a temperature of the treatment chamber is maintained in a range of 18 to 22° C.

In some aspects of the method, a temperature of the treatment chamber is maintained at 20° C.

In some aspects, the techniques described herein relate to a method for ultrasonic antimicrobial treatment of camel milk, including introducing raw camel milk into a treatment chamber, subjecting the raw camel milk to ultrasonic vibrations within the treatment chamber, and evacuating treated camel milk from the treatment chamber.

The method of the preceding paragraph can optionally include, additionally and/or alternatively any, one or more of the following features/steps, configurations and/or additional components.

In some aspects of the method, the raw camel milk is continually introduced into the treatment chamber and the treated camel milk is continually evacuated from the treatment chamber.

In some aspects of the method, introduction of camel milk into the treatment chamber and evacuation of camel milk from the treatment chamber is performed at a constant flow rate.

In some aspects of the method, the ultrasonic vibrations have a frequency of about 20 kHz.

In some aspects of the method, the raw camel milk is subjected to the ultrasonic vibrations in a pulsed mode with a 50% duty cycle for a period of at least 10 minutes.

In some aspects of the method, each duty cycle lasts 20 seconds and includes 10 seconds of applying the ultrasonic vibrations followed by 10 seconds of not the applying ultrasonic vibrations.

In some aspects of the method, the ultrasonic vibrations have a power of about 750 watts.

In some aspects of the method, the flow rate allows the raw camel milk to be subjected to the ultrasonic vibrations for about 10 minutes.

In some aspects of the method, a temperature of the treatment chamber is maintained in a range of 18 to 22° C.

In some aspects of the method, a temperature of the treatment chamber is maintained at 20° C.

1. A method of producing yogurt comprising:
pasteurizing a volume of milk;
adding at least one additive to form a milk mixture, wherein the at least one additive comprises camel whey protein hydrolysate; and
fermenting the milk mixture.

2. The method of claim 1, wherein the camel whey protein hydrolysate is 5% of the milk mixture.

3. The method of claim 1, the at least one additive further comprises at least one coagulant, and/or at least one prebiotic.

4. The method of claim 1, wherein the at least one additive further comprises 1% v/v/ bottle gourd seed extract per 100 mL of heat-treated milk.

5. The method of claim 4, wherein the at least one additive further comprises 1% v/v artichoke extract per 100 mL of heat-treated milk.

6. The method of claim 1, wherein fermenting the milk mixture comprises:
adding P. acidilactici bacteria to the milk mixture, wherein the P. acidilactici bacteria is non-hemolytic and characterized by one or more of:
a survival rate in pH≤2.5 of approximately 86%-96%;
a survival rate in pepsin of approximately 66%-76%; or
a survival rate in bile salts and pancreatin of approximately 65%-75%.

7. The method of claim 6, wherein the P. acidilactici bacteria is further characterized by one or more of:
a bile salt hydrolase (BSH) activity characterized by a precipitation zone of approximately 15 mm-17 mm; or
a cholesterol assimilation of approximately 80%-89%.

8. The method of claim 6, wherein the P. acidilactici bacteria is further characterized by resistance to a plurality of antibiotics selected from the group consisting of ampicillin, streptomycin, penicillin, tetracycline, kanamycin, erythromycin, gentamicin, clindamycin, and chloramphenicol.

9. The method of claim 6, wherein the P. acidilactici bacteria is P. acidilactici MNL2.

10. The method of claim 1, wherein pasteurizing the volume of milk comprises:
heating the volume of milk at a pasteurization temperature for a pasteurization time period;
cooling the volume of milk to a second temperature; and
maintaining volume of milk at the second temperature for a third time period.

11. The method of claim 10, wherein the pasteurization temperature is approximately 60° C.-70° C.

12. The method of claim 10, wherein the pasteurization temperature is 65° C., the pasteurization time period is 30 minutes, the second temperature is 38° C.; and the third time period is 10 minutes.

13. The method of claim 10, wherein fermenting the milk mixture further comprises incubating at a fermentation temperature until a pH of the fermentation mixture reaches 4.5.

14. The method of claim 1, wherein the milk is bovine, sheep, goat, pig, mouse, water buffalo, camel, yak, horse, donkey, llama, or human.

15. A method of producing yogurt comprising:
pasteurizing a volume of milk;
adding at least one additive to form a milk mixture; and
fermenting the milk mixture after adding P. acidilactici bacteria to the milk mixture, wherein the P. acidilactici bacteria are non-hemolytic and are characterized by one or more of:
a survival rate in pH≤2.5 of approximately 86%-96%;
a survival rate in pepsin of approximately 66%-76%; or
a survival rate in bile salts and pancreatin of approximately 65%-75%.

16. The method of claim 15, wherein the P. acidilactici bacteria are further characterized by one or more of:
a bile salt hydrolase (BSH) activity characterized by a precipitation zone of approximately 15 mm-17 mm; or
a cholesterol assimilation of approximately 80%-89%.

17. The method of claim 16, wherein the P. acidilactici bacteria is further characterized by resistance to a plurality of antibiotics selected from the group consisting of ampicillin, streptomycin, penicillin, tetracycline, kanamycin, erythromycin, gentamicin, clindamycin, and chloramphenicol.

18. The method of claim 15, wherein the P. acidilactici bacteria is P. acidilactici MNL2.

19. A fermented milk product comprising:
active P. acidilactici bacteria that are non-hemolytic and characterized by one or more of:
a survival rate in pH≤2.5 of approximately 86%-96%;
a survival rate in pepsin of approximately 66%-76%; or
a survival rate in bile salts and pancreatin of approximately 65%-75%;
wherein the fermented milk product is yogurt.

20. The fermented milk product of claim 19, where the P. acidilactici bacteria are P. acidilactici MNL2.
